(19) United States
(12) Patent Application Publication
(10) Pub. No.: US 2017/0071470 A1
(43) Pub. Date: Mar. 16, 2017

(54) Framework for Abnormality Detection in Multi-Contrast Brain Magnetic Resonance Data

(71) Applicant: Siemens Healthcare GmbH, Erlangen (DE)

(72) Inventors: Hasan E. Cetingul, Baltimore, MD (US); Benjamin L. Odry, West New York, NJ (US); Mariappan S. Nadar, Plainsboro, NJ (US)

(21) Appl. No.: 14/854,330

(22) Filed: Sep. 15, 2015

(51) Int. Cl. A61B 5/00; G06F 19/00; A61B 5/055

(52) U.S. Cl. CPC A61B 5/0042; A61B 5/055; G06F 19/3437

(57) ABSTRACT

A computer-implemented method for identifying abnormalities in Magnetic Resonance (MR) brain image data includes a computer receiving multi-contrast MR image data of a subject's brain and identifying, within the multi-contrast MR image data, (i) an abnormality region comprising one or more suspected abnormalities and (ii) a healthy region comprising healthy tissue. The computer creates a model of the healthy region, computes a novelty score for each voxel in the multi-contrast MR image data based on the abnormality region and the model, and creates an abnormality map of the subject's brain based on the novelty score computed for each voxel in the multi-contrast MR image data.

### TECHNOLOGY FIELD

The present invention relates generally to methods, systems, and apparatuses for detecting abnormalities in multi-contrast Magnetic Resonance Imaging (MRI) brain data. The disclosed techniques may be applied to, for example, the detection of multiple sclerosis (MS), traumatic brain injury, ischemic stroke, and atypical gliomas.

### BACKGROUND

The problem of automatically detecting abnormalities (e.g., pathologies in the form of tumors, lesions, or structures such as metal implants, etc.) in imaging data has been a topic of interest for several years. In particular, detection and delineation of diffusely abnormal lesions (e.g., hyper-intensities encountered in the brain images of patients with multiple sclerosis, traumatic brain injuries) has gained pace since the advent of today's multi-contrast (e.g., T1/T2/PD/FLAIR/SWI) MR imaging protocols. However, this problem is challenging due to the complex manifestation of pathology (e.g., highly diffuse vs. somehow focal spatial profiles, highly variable contrast profiles, etc.) in the acquired images. Hence, manual delineation is still a common practice in the clinic. In addition, state-of-the-art medical image analysis approaches often employ supervised learning schemes that require carefully designed features/biomarkers and a considerable amount of training data for “robustness.” These approaches may not promote a patient-specific testbed, and they, implicitly or explicitly, aim to infer a model or representation for abnormality, which may not be necessary.

Novelty detection (ND), also referred to as abnormality or outlier detection in the literature, has been a topic of interest to the researchers for more than two decades. The existing novelty detection techniques can be categorized into probabilistic, distance-based, domain-based, reconstruction-based, and information theoretic ND, with applications ranging from IT security, text mining, industrial monitoring and damage detection to healthcare informatics and medical diagnostics and monitoring.

Accordingly, given the capabilities available in ND, it is desired to provide a framework for applying ND in the context of detecting abnormalities in multi-contrast Magnetic Resonance Imaging (MRI) brain data.

### SUMMARY

Embodiments of the present invention address and overcome one or more of the above shortcomings and drawbacks, by providing methods, systems, and apparatuses related to a complete medical image analysis framework that detects abnormalities in multi-contrast brain MR data. The disclosed techniques may be applied, for example, to the diagnostic/prognostic imaging of brain abnormalities. Possible clinical uses include, without limitation, multiple sclerosis, traumatic brain injury, ischemic stroke, and atypical gliomas. The extensions of the techniques described herein can also be used for tumor detection in other organs such as the liver and lungs.

According to some embodiments of the present invention, computer-implemented method for identifying abnormalities in Magnetic Resonance (MR) brain image data includes a computer receiving multi-contrast MR image data of a subject's brain and identifying, within the multi-contrast MR image data, (i) an abnormality region comprising one or more suspected abnormalities and (ii) a healthy region comprising healthy tissue. The computer creates a model of the healthy region, computes a novelty score for each voxel in the multi-contrast MR image data based on the abnormality region and the model, and creates an abnormality map of the subject's brain based on the novelty score computed for each voxel in the multi-contrast MR image data.

The features of the aforementioned method may be refined, enhanced, supplemented, or otherwise modified in different embodiments of the present invention. For example, in some embodiments, prior to identifying the abnormality region and the healthy region, one or more image pre-processing procedures are applied to the multi-contrast MR image data. These image pre-processing procedures may include, for example, an inhomogeneity correction procedure, a motion correction procedure, a skull stripping procedure, a resampling procedure, a filtering/denoising procedure, and/or a high-level tissue segmentation procedure. In some embodiments, the novelty score is computed for each voxel using an analytical multivariate extreme value theory (EVT) approximation. In some embodiments, the aforementioned method further comprises identifying voxels in the multi-contrast MR image data corresponding to novelty scores above a predetermined threshold value. These voxels may then be used to depict abnormalities in the abnormality map. In some embodiments, one or more anatomical masks may be used to identify false positive voxels in the abnormality map. These false positive voxels may then be identified as healthy tissue in the abnormality map.

The process of defining the abnormality region in the aforementioned method may also vary in different embodiments. In some embodiments, the abnormality region is defined by bounding box manually drawn by a user using a graphical user interface operably coupled to the computer. In other embodiments, the abnormality region is defined by bounding box automatically generated by the computer using an unsupervised change detection method that searches for a most dissimilar region in the left and right halves of the subject's brain. In still other embodiments, the abnormality region is defined by the computer using a fully automated procedure that analyzes the multi-contrast MR image data and generates a list of voxels that are suspected to be abnormal.

Various types of parametric and non-parametric models may be used in the aforementioned method. For example, in some embodiments, a Gaussian mixture model (GMM) is used. In these embodiments, the fully automated procedure discussed above may comprise fitting a GMM via expectation maximization (EM) to the multi-contrast MR image data over a plurality of iterations. Each voxel of the multi-contrast MR image data is checked during each iteration of this fully automated procedure to determine whether it should be placed in the abnormality region or the healthy region.

According to other embodiments of the present invention, an article of manufacture for identifying abnormalities in MR brain image data comprises a non-transitory, tangible computer-readable medium holding computer-executable instructions for performing the aforementioned method, with or without the additional features discussed above.

According to other embodiments, a system for identifying abnormalities in MR brain image data comprises an imaging device and a computer. The imaging device is configured to acquire multi-contrast MR image data of a subject's brain. The computer comprises one or more processors which are configured to: identify, within the multi-contrast MR image data, (i) an abnormality region comprising one or more suspected abnormalities and (ii) a healthy region comprising healthy tissue, create a model of the healthy region, compute a novelty score for each voxel in the multi-contrast MR image data based on the abnormality region and the model, and create an abnormality map of the subject's brain based on the novelty score computed for each voxel in the multi-contrast MR image data.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 shows a system for ordering acquisition of frequency domain components representing magnetic resonance image data for storage in a k-space storage array, as used by some embodiments of the present invention;

FIG. 2 provides an illustration of an image analysis framework, according to some embodiments of the present invention;

FIG. 3 shows examples of bounding boxes, as applied to the automated localization of ischemic stroke in typical diffusion and perfusion maps;

FIG. 4 provides a flowchart showing the process performed at step 210D in FIG. 2, according to some embodiments;

FIG. 5A shows the input image data, the simulated lesion, and the abnormal regions given by the framework shown in FIG. 2;

FIG. 5B shows the input images, the annotated lesion, and the results given by the framework shown in FIG. 2 at different levels of novelty score threshold;

FIG. 6 shows the input images, the binary mask used to exclude the suspected abnormality, and the abnormal regions given by our framework; and

FIG. 7 illustrates an exemplary computing environment within which embodiments of the invention may be implemented.

### DETAILED DESCRIPTION OF ILLUSTRATIVE EMBODIMENTS

The following disclosure describes the present invention according to several embodiments directed at methods, systems, and apparatuses related to a complete medical image analysis framework that detects abnormalities in multi-contrast brain Magnetic Resonance (MR) data. More specifically, the medical image analysis framework described herein accepts multi-contrast (T1/T2/PD/FLAIR/SWI) brain MR data of a single subject, identifies normal tissues with or without user guidance, performs parametric modeling of these normal tissues, and applies a slightly modified version of the novelty detection (ND) with multivariate extreme value theory (EVT) to the entire image data in order to detect abnormalities, if present, in the subject's brain. This framework may be applied to, for example, the detection of multiple sclerosis, traumatic brain injury, ischemic stroke, and atypical gliomas and therefore, may be used to monitor treatment.

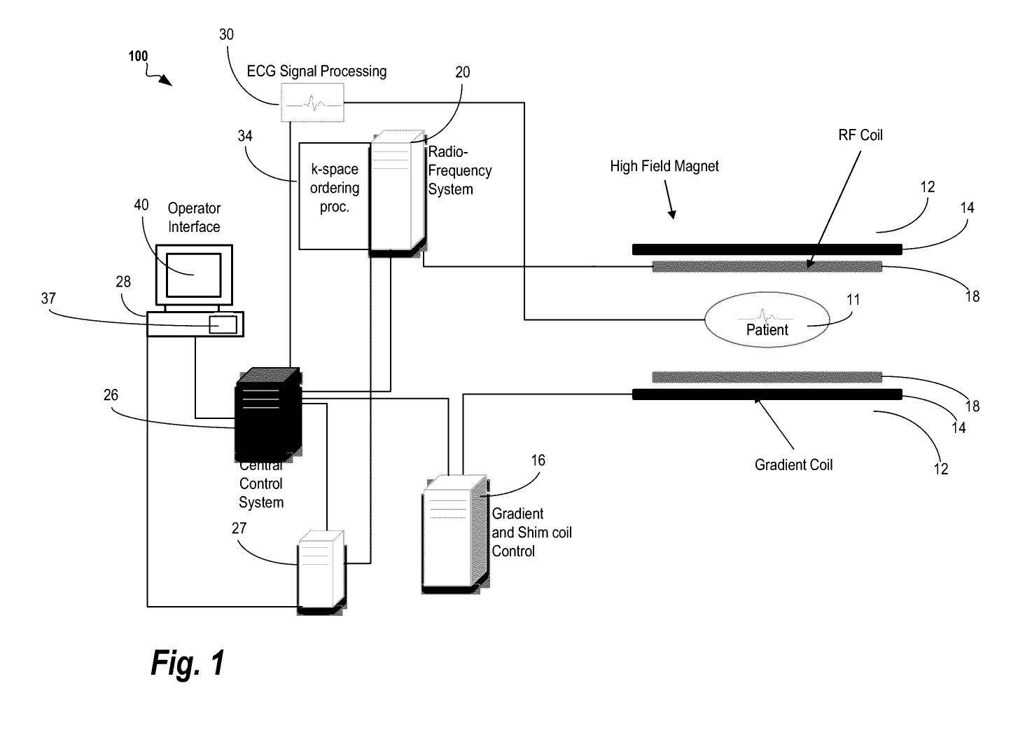

FIG. 1 shows a system 100 for ordering acquisition of frequency domain components representing MRI data for storage in a k-space storage array, as used by some embodiments of the present invention. In system 100, magnetic coils 18 create a static base magnetic field in the body of patient 11 to be imaged and positioned on a table. Within the magnet system are gradient coils 14 for producing position dependent magnetic field gradients superimposed on the static magnetic field. Gradient coils 14, in response to gradient signals supplied thereto by a gradient and shim coil control module 16, produce position dependent and shimmed magnetic field gradients in three orthogonal directions and generates magnetic field pulse sequences. The shimmed gradients compensate for inhomogeneity and variability in an MRI device magnetic field resulting from patient anatomical variation and other sources. The magnetic field gradients include a slice-selection gradient magnetic field, a phase-encoding gradient magnetic field and a readout gradient magnetic field that are applied to patient 11.

Further radio frequency (RF) module 20 provides RF pulse signals to RF coil 18, which in response produces magnetic field pulses which rotate the spins of the protons in the imaged body of the patient 11 by ninety degrees or by one hundred and eighty degrees for so-called “spin echo” imaging, or by angles less than or equal to 90 degrees for so-called “gradient echo” imaging. Gradient and shim coil control module 16 in conjunction with RF module 20, as directed by central control unit 26, control slice-selection, phase-encoding, readout gradient magnetic fields, radio frequency transmission, and magnetic resonance signal detection, to acquire magnetic resonance signals representing planar slices of patient 11.

In response to applied RF pulse signals, the RF coil 18 receives magnetic resonance signals, i.e., signals from the excited protons within the body as they return to an equilibrium position established by the static and gradient magnetic fields. The magnetic resonance signals are detected and processed by a detector within RF module 20 and k-space component processor unit 34 to provide a magnetic resonance dataset to an image data processor for processing into an image. In some embodiments, the image data processor is located in central control unit 26. However, in other embodiments such as the one depicted in FIG. 1, the image data processor is located in a separate unit 27. Electrocardiogram (ECG) synchronization signal generator 30 provides ECG signals used for pulse sequence and imaging synchronization. A two or three dimensional k-space storage array of individual data elements in k-space component processor unit 34 stores corresponding individual frequency components comprising a magnetic resonance dataset. The k-space array of individual data elements has a designated center and individual data elements have a radius to the designated center.

A magnetic field generator (comprising coils 18, 14, and 18) generates a magnetic field for use in acquiring multiple individual frequency components corresponding to individual data elements in the storage array. The individual frequency components are successively acquired in an order in which the radius of respective corresponding individual data elements increases and decreases along a substantially spiral path as the multiple individual frequency components is sequentially acquired during acquisition of a magnetic resonance dataset representing an magnetic resonance image. A storage processor in the k-space component processor unit 34 stores individual frequency components acquired using the magnetic field in corresponding individual data elements in the array. The radius of respective corresponding individual data elements alternately increases and decreases as multiple sequential individual frequency components are acquired. The magnetic field acquires individual frequency components in an order corresponding to a sequence of substantially adjacent individual data elements in the array and magnetic field gradient change between successively acquired frequency components is substantially minimized.

Central control unit 26 uses information stored in an internal database to process the detected magnetic resonance signals in a coordinated manner to generate high quality images of a selected slice(s) of the body (e.g., using the image data processor) and adjusts other parameters of system 100. The stored information comprises predetermined pulse sequence and magnetic field gradient and strength data as well as data indicating timing, orientation and spatial volume of gradient magnetic fields to be applied in imaging. Generated images are presented on display 40 of the operator interface. Computer 28 of the operator interface includes a graphical user interface (GUI) enabling user interaction with central control unit 26 and enables user modification of magnetic resonance imaging signals in substantially real time. Continuing with reference to FIG. 1, display processor 37 processes the magnetic resonance signals to reconstruct one or more images for presentation on display 40, for example. Various techniques may be used for reconstruction. For example, as described in greater detail below, an optimization algorithm is applied to iteratively solve a cost function which results in the reconstructed image.

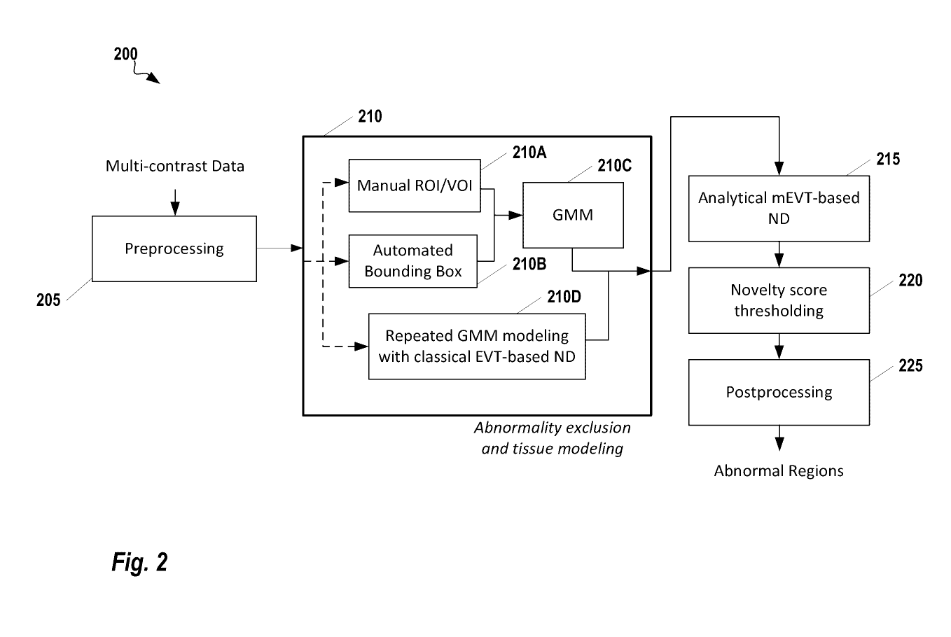

FIG. 2 provides an illustration of an image analysis framework 200, according to some embodiments of the present invention. Multi-contrast MR data is acquired, for example, using the system 100 illustrated in FIG. 1. The type of data may vary according to the clinical application. For example, in the case of MS lesion detection, the multi-contrast MR data may be T1/T2/PD or T1/T2/FLAIR data. For analyzing ischemic stroke, perfusion and diffusion maps (e.g., Cerebral Blood Volume, Cerebral Blood Flow, Mean Transit Time, Time to Peak, with Apparent Diffusion Coefficient and/or Trace Weighted Images) in the case of analyzing ischemic stroke may be used. At step 205, image preprocessing steps are applied to improve the quality of the acquired multi-contrast MR data. These image pre-processing steps may include, for example, inhomogeneity correction, motion correction, skull stripping, resampling, filtering/denoising, high-level tissue segmentation, etc. For example, in one embodiment, clinical priors about the pathology under study (e.g., MS lesions occur in white matter) and certain structural MR images such as T1, T2, and/or proton-density (PD) may be used to apply gray matter (GM), white matter (WM), and/or cerebrospinal fluid (CSF) segmentation to the image data to obtain a coarse outline of these brain tissues. The resulting segmentations, along with the skull stripped images, may be used, if needed, as binary masks.

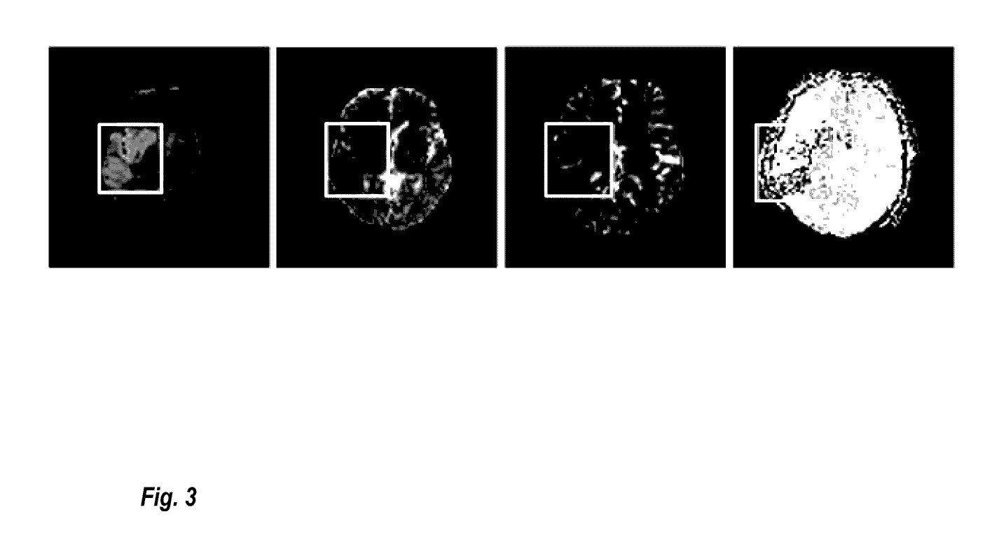

Continuing with reference to FIG. 2, at step 210, regions of the image with suspected abnormalities are excluded. Dashed lines in FIG. 2 denote optional routes. In some embodiments, this exclusion is performed by manually drawing (e.g., via a graphical user interface) or automatically placing a single or multiple bounding boxes that loosely encapsulate the suspected abnormality. This is depicted by steps 210A and 210B respectively. For the Automated Bounding Box technique (i.e., step 210B), a bounding box is placed to surround relatively large and focal intracranial masses by using intensity information about the left-right brain symmetry. This method can be considered as an unsupervised change detection method that searches for the most dissimilar region between the left and right halves of a brain: it places an axis-parallel bounding box by finding the extrema of a score function based on the Bhattacharya coefficient computed from the gray-level intensity histograms. As is well understood in the art, the Bhattacharya coefficient provides an approximate measurement of the amount of overlap between two statistical samples. FIG. 3 shows examples of bounding boxes given by step 210B, as applied to the automated localization of ischemic stroke in typical diffusion and perfusion maps.

After exclusion at step 210A or 210B, at step 210C, the remaining normal tissues are modeled using Gaussian mixture models (GMMs). The GMM may be parameterized according to the particular contrast being employed (e.g., number of mixture components, K=3 for GM/WM/CSF; feature dimension, d=3 for T1/T2/PD).

In other embodiments, the regions of the image with suspected abnormalities are excluded using a fully automated mechanism that analyzes the entire imaging data and generates a list of voxels that are suspected, with some level of certainty, to be abnormal. This is illustrated by step 210D in FIG. 2. This step 210D applies Gaussian mixture modeling via expectation maximization (EM) multiple times and checks at each iteration whether a voxel belongs to a normal tissue/region (GM, WM, or CSF) or to an “abnormality” that is probabilistically described via a classical extreme value distribution, i.e., the Gumbel distribution, with pdf f_{Gumbel} (•; c, d). Here, c and d are parameters that depend on the amount of data sampled and on the closest normal distribution.

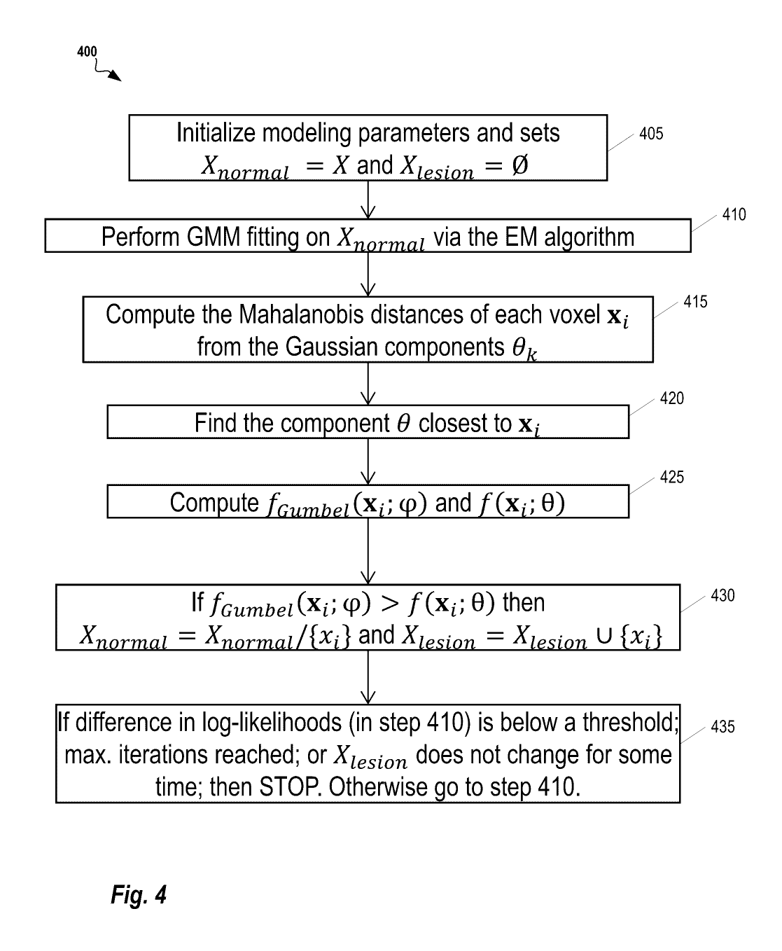

FIG. 4 provides a flowchart showing the process 400 performed at step 210D in FIG. 2, according to some embodiments. At step 405, the parameters (the number of mixture components K and the dimension d) are initialized according to the data sources. For example, K=3 GM, WM, and CSF are used as tissues; d=3 if T1, T2 and PD are used as image volumes. Additionally, the value of Xₙₒᵣₘₐₗ is set to the initial image X and Xₗₑₛᵢₒₙ is set to 0. At step 410, the EM algorithm is performed on Xₙₒᵣₘₐₗ to classify each voxel i of the brain into K different classes by using the multi-contrast (intensity) profiles as input vectors xᵢ=[T1ᵢ, T2ᵢ, PDᵢ]. EM computes the parameters θₖ={μₖ, Σₖ} for class k=1,2, . . . , K.

Next, at step 415, the Mahalanobis distances of each voxel i from the Gaussian components θₖ are computed. At step 420, the component closest to voxel i is located and denoted by k*. Then, at step 425, with the sets θ={μ_{k*}, Σ_{k*}} and φ={c_{k*}, d_{k*}} denoting the parameters related to the k*-th component, f(xᵢ; θ) and f_{Gumbel}(xᵢ; φ) are computed. At step 430, if f_{Gumbel}(xᵢ; φ)>f(xᵢ; θ), then voxel i is considered a candidate WM lesion. It may then be removed from the set of all data points Xₙₒᵣₘₐₗ and stored as an element of a new set Xₗₑₛᵢₒₙ. These two sets are updated every time a voxel is found to be a candidate WM lesion. Parametric modeling of normal tissues is automatically performed during the search for candidate voxels for abnormality. As shown in step 435, if the differences in log-likelihoods (in step 410) is below a threshold; Xₗₑₛᵢₒₙ has not changed for several iterations; or the maximum number of iterations is reached, the process 400 ends. Otherwise, the process 400 repeats for a second iteration at step 410, considering the continuously updated set Xₙₒᵣₘₐₗ as input. The final set Xₗₑₛᵢₒₙ contains candidate WM lesion voxels.

Returning to FIG. 2, at step 215, with normal tissues modeled via GMMs, an analytical multivariate EVT approximation is applied to compute a probabilistically meaningful novelty score for each voxel in the image data. It starts by recursively computing a distribution Gₙ over the underlying n-variate pdf fₙ:

Gₙ(y)=∫_{f}_{ₙ}_{^{−1}}_{(]0,y])}fₙ(x)dx, ∀yεP.

Here, y denotes the variable in the probability space P=[0,1]. One can determine the extreme value distribution (EVD) in the data space by determining the EVD of Gₙ in P. Using this observation, after some recursive formulation, one can find an approximation to the EVD as

Gₙ^{e}(y)=1−e^{(-y/c}^{ₘ}⁾^{^{α}}^{m},

and the novelty score is computed as

$${F}_{n}^{e} ( x ) = 1 - {G}_{n}^{e} ( {f}_{n} ( x ) ) = {e}^{- {( \frac{1}{{C}_{n} {c}_{m}} {e}^{- \frac{{M ( x )}^{2}}{2}} )}^{{α}_{m}}} .$$

Here, cₘ and αₘ are the parameters of the EVD, Cₙ is a constant, and M(x) is the Mahalanobis distance of the data vector x and its closest Gaussian component θ_{k*}, i.e., M(x)=\sqrt{(x−μ_{k*})T Σ_{k*}⁻¹(x−μ_{k*})}. At step 220, the set of voxels {i:Fₙ^{e}(xᵢ)>t} for some user-specified threshold t are found as “abnormal.” Typical values for t are in the range [0.8,1−ε] for a very small ε>0. Finally at step 225, the abnormality map is post-processed using anatomical masks and/or morphological operations to eliminate the false positives. At this point, an image, referred to herein as an “abnormality map” can be generated which shows the abnormal regions of the brain (e.g., highlighted using colors or other visual indicators to distinguish normal and abnormal tissue).

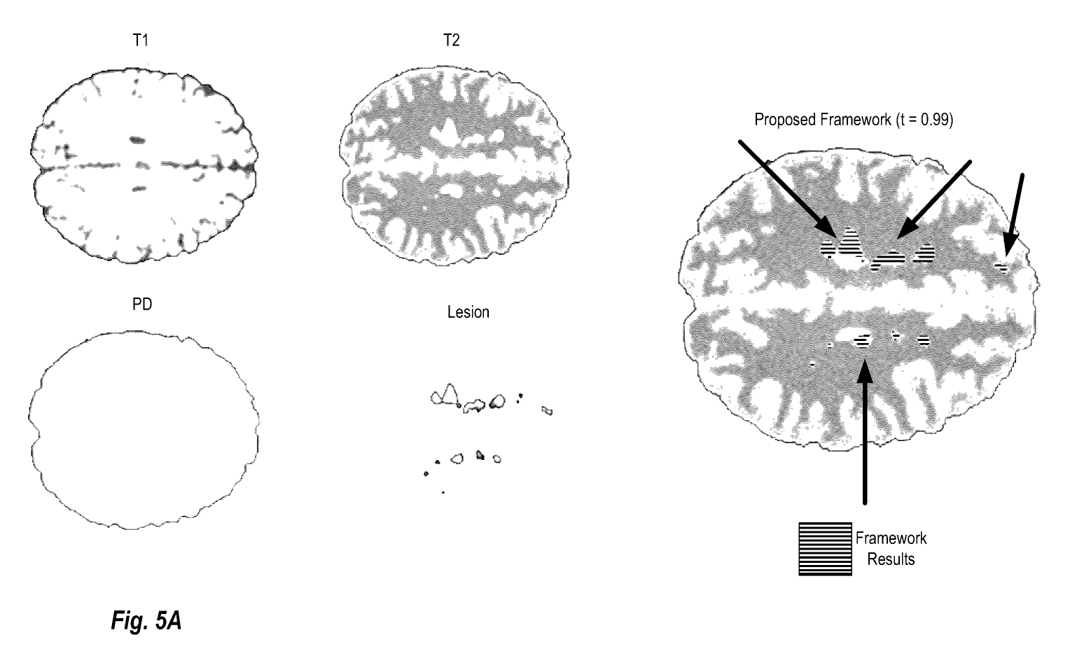

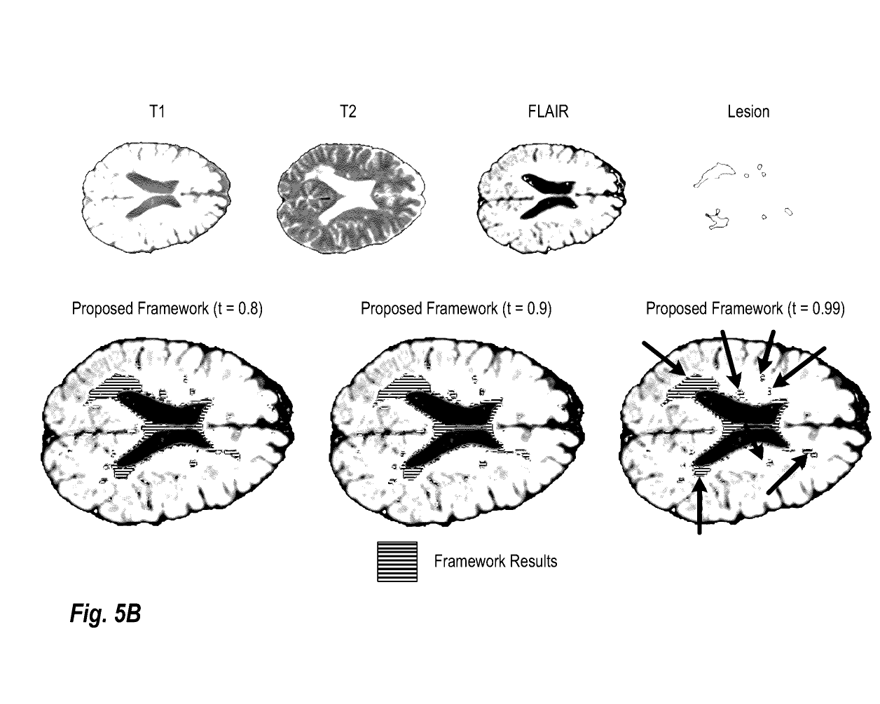

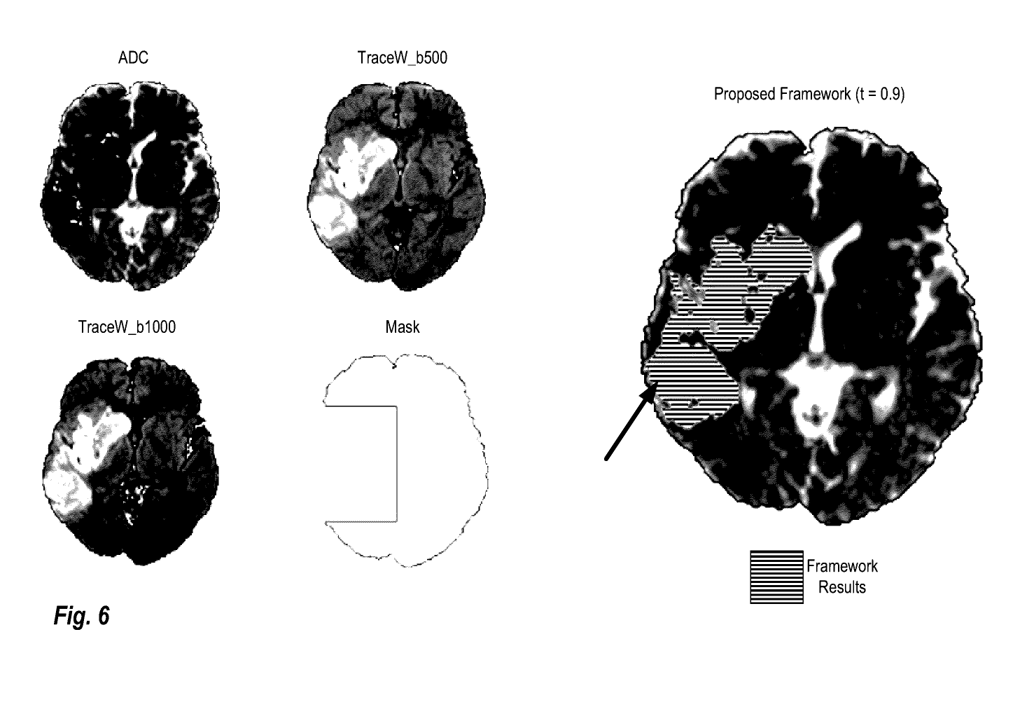

To illustrate the applicability of the framework described above with reference to FIG. 2, the framework was evaluated on multiple datasets including those related to MS lesions and ischemic stroke. The results of these evaluations are discussed below with reference to the abnormality maps displayed in FIGS. 5A, 5B, and 6. In each of these images, the results of the framework are presented in stripes and indicated by one or more arrows.

Regarding MS lesions, the initial evaluations were performed on the BrainWeb dataset where MS lesions with different levels (mild, medium, severe) are simulated in T1, T2, and PD image volumes. FIG. 5A shows the input image data, the simulated lesion, and the abnormal regions given by the framework shown in FIG. 2. The detected abnormal regions are found to overlap very well with the ground-truth lesion. Additional experiments were performed on the MSGC08 dataset, containing several annotated and non-annotated MS lesion data for the MS Grand Challenge at MICCAI'08. FIG. 5B shows the input images, the annotated lesion, and the results given by the framework shown in FIG. 2 at different levels of novelty score threshold. It is observed that the detected region is a superset of the lesion, but further post-processing is required to eliminate false positives.

The framework shown in FIG. 2 was also tested on a number of diffusion maps (Apparent Diffusion Coefficient images, Trace Weighted images at different b-values) for stroke segmentation. FIG. 6 shows the input images, the binary mask used to exclude the suspected abnormality, and the abnormal regions given by our framework. It is observed that the segmented region delineates very well the extent of pathology.

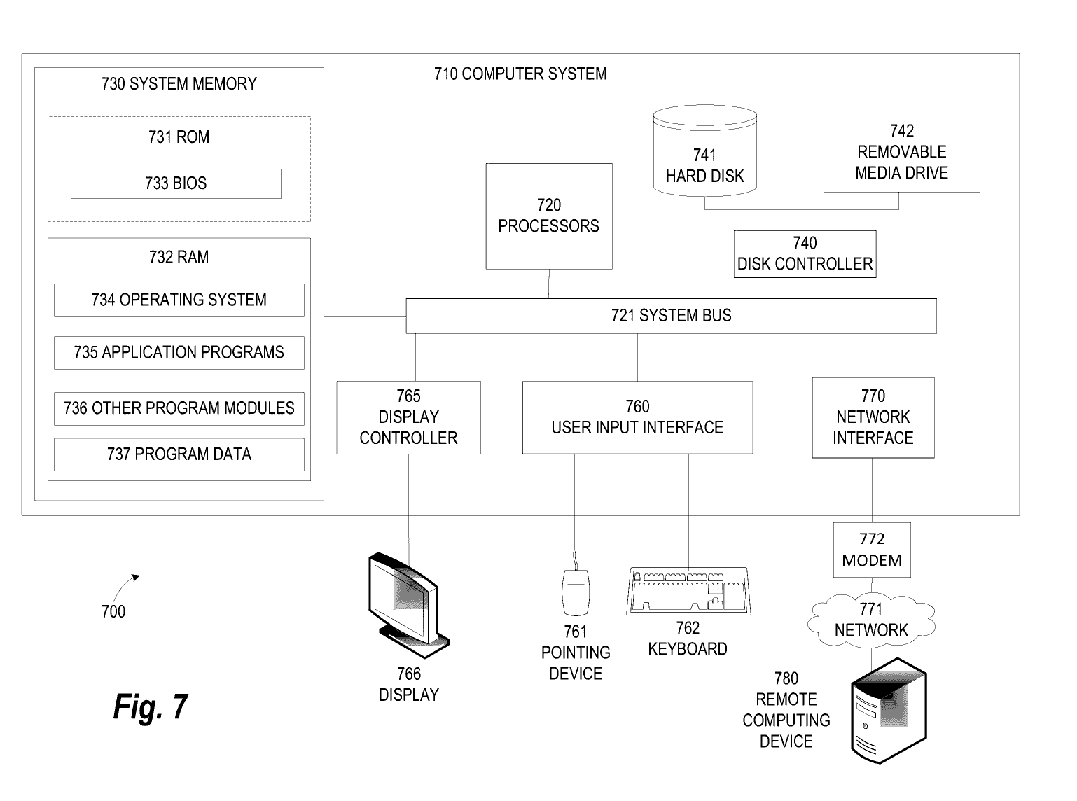

FIG. 7 illustrates an exemplary computing environment 700 within which embodiments of the invention may be implemented. For example, this computing environment 700 may be used to implement the framework 200 described in FIG. 2. In some embodiments, the computing environment 700 may be used to implement one or more of the components illustrated in the system 100 of FIG. 1. The computing environment 700 may include computer system 710, which is one example of a computing system upon which embodiments of the invention may be implemented. Computers and computing environments, such as computer system 710 and computing environment 700, are known to those of skill in the art and thus are described briefly here.

As shown in FIG. 7, the computer system 710 may include a communication mechanism such as a bus 721 or other communication mechanism for communicating information within the computer system 710. The computer system 710 further includes one or more processors 720 coupled with the bus 721 for processing the information. The processors 720 may include one or more central processing units (CPUs), graphical processing units (GPUs), or any other processor known in the art.

The computer system 710 also includes a system memory 730 coupled to the bus 721 for storing information and instructions to be executed by processors 720. The system memory 730 may include computer readable storage media in the form of volatile and/or nonvolatile memory, such as read only memory (ROM) 731 and/or random access memory (RAM) 732. The system memory RAM 732 may include other dynamic storage device(s) (e.g., dynamic RAM, static RAM, and synchronous DRAM). The system memory ROM 731 may include other static storage device(s) (e.g., programmable ROM, erasable PROM, and electrically erasable PROM). In addition, the system memory 730 may be used for storing temporary variables or other intermediate information during the execution of instructions by the processors 720. A basic input/output system (BIOS) 733 containing the basic routines that help to transfer information between elements within computer system 710, such as during start-up, may be stored in ROM 731. RAM 732 may contain data and/or program modules that are immediately accessible to and/or presently being operated on by the processors 720. System memory 730 may additionally include, for example, operating system 734, application programs 735, other program modules 736 and program data 737.

The computer system 710 also includes a disk controller 740 coupled to the bus 721 to control one or more storage devices for storing information and instructions, such as a hard disk 741 and a removable media drive 742 (e.g., floppy disk drive, compact disc drive, tape drive, and/or solid state drive). The storage devices may be added to the computer system 710 using an appropriate device interface (e.g., a small computer system interface (SCSI), integrated device electronics (IDE), Universal Serial Bus (USB), or FireWire).

The computer system 710 may also include a display controller 765 coupled to the bus 721 to control a display 766, such as a cathode ray tube (CRT) or liquid crystal display (LCD), for displaying information to a computer user. The computer system includes an input interface 760 and one or more input devices, such as a keyboard 762 and a pointing device 761, for interacting with a computer user and providing information to the processor 720. The pointing device 761, for example, may be a mouse, a trackball, or a pointing stick for communicating direction information and command selections to the processor 720 and for controlling cursor movement on the display 766. The display 766 may provide a touch screen interface which allows input to supplement or replace the communication of direction information and command selections by the pointing device 761.

The computer system 710 may perform a portion or all of the processing steps of embodiments of the invention in response to the processors 720 executing one or more sequences of one or more instructions contained in a memory, such as the system memory 730. Such instructions may be read into the system memory 730 from another computer readable medium, such as a hard disk 741 or a removable media drive 742. The hard disk 741 may contain one or more datastores and data files used by embodiments of the present invention. Datastore contents and data files may be encrypted to improve security. The processors 720 may also be employed in a multi-processing arrangement to execute the one or more sequences of instructions contained in system memory 730. In alternative embodiments, hard-wired circuitry may be used in place of or in combination with software instructions. Thus, embodiments are not limited to any specific combination of hardware circuitry and software.

As stated above, the computer system 710 may include at least one computer readable medium or memory for holding instructions programmed according to embodiments of the invention and for containing data structures, tables, records, or other data described herein. The term “computer readable medium” as used herein refers to any medium that participates in providing instructions to the processor 720 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 741 or removable media drive 742. Non-limiting examples of volatile media include dynamic memory, such as system memory 730. Non-limiting examples of transmission media include coaxial cables, copper wire, and fiber optics, including the wires that make up the bus 721. Transmission media may also take the form of acoustic or light waves, such as those generated during radio wave and infrared data communications.

The computing environment 700 may further include the computer system 710 operating in a networked environment using logical connections to one or more remote computers, such as remote computer 780. Remote computer 780 may be a personal computer (laptop or desktop), a mobile device, a server, a router, a network PC, a peer device or other common network node, and typically includes many or all of the elements described above relative to computer system 710. When used in a networking environment, computer system 710 may include modem 772 for establishing communications over a network 771, such as the Internet. Modem 772 may be connected to bus 721 via user network interface 770, or via another appropriate mechanism.

Network 771 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 710 and other computers (e.g., remote computer 780). The network 771 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 771.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112, sixth paragraph, unless the element is expressly recited using the phrase “means for.”

We claim:

1. A computer-implemented method for identifying abnormalities in Magnetic Resonance (MR) brain image data, the method comprising:
receiving, by a computer, multi-contrast MR image data of a subject's brain;
identifying, within the multi-contrast MR image data, (i) an abnormality region comprising one or more suspected abnormalities and (ii) a healthy region comprising healthy tissue;
creating, by the computer, a model of the healthy region;
computing, by the computer, a novelty score for each voxel in the multi-contrast MR image data based on the abnormality region and the model; and
creating, by the computer, an abnormality map of the subject's brain based on the novelty score computed for each voxel in the multi-contrast MR image data.

2. The method of claim 1, further comprising:
prior to identifying the abnormality region and the healthy region, applying one or more image pre-processing procedures to the multi-contrast MR image data.

3. The method of claim 2, wherein the one or more image pre-processing procedures comprise one or more of an inhomogeneity correction procedure, a motion correction procedure, a skull stripping procedure, a resampling procedure, a filtering/denoising procedure or a high-level tissue segmentation procedure.

4. The method of claim 1, wherein the abnormality region is defined by bounding box manually drawn by a user using a graphical user interface operably coupled to the computer.

5. The method of claim 1, wherein the abnormality region is defined by bounding box automatically generated by the computer using an unsupervised change detection method that searches for a most dissimilar region left and right halves of the subject's brain.

6. The method of claim 1, wherein the abnormality region is defined by the computer using a fully automated procedure that analyzes the multi-contrast MR image data and generates a list of voxels that are suspected to be abnormal.

7. The method of claim 6, wherein the fully automated procedure comprises:
fitting a Gaussian mixture model (GMM) via expectation maximization (EM) to the multi-contrast MR image data over a plurality of iterations, wherein each voxel of the multi-contrast MR image data is checked during each iteration of the fully automated procedure to determine whether it should be placed in the abnormality region or the healthy region.

8. The method of claim 1, wherein the model comprises a parametric model.

9. The method of claim 8, wherein the parametric model comprises a Gaussian mixture model (GMM).

10. The method of claim 1, wherein the model comprises a non-parametric model.

11. The method of claim 1, wherein the novelty score is computed for each voxel using an analytical multivariate extreme value theory (EVT) approximation.

12. The method of claim 1, further comprising:
identifying a plurality of voxels in the multi-contrast MR image data corresponding to novelty scores above a predetermined threshold value,
wherein the abnormality map depicts abnormalities at the plurality of voxels.

13. The method of claim 1, further comprising:
using one or more anatomical masks to identify one or more false positive voxels in the abnormality map; and
identifying the one or more false positive voxels as healthy tissue in the abnormality map.

14. An article of manufacture for identifying abnormalities in Magnetic Resonance (MR) brain image data, the article of manufacture comprising a non-transitory, tangible computer-readable medium holding computer-executable instructions for performing a method comprising:
identifying, within multi-contrast MR image data of a subject's brain, (i) an abnormality region comprising one or more suspected abnormalities and (ii) a healthy region comprising healthy tissue;
creating a model of the healthy region;
computing a novelty score for each voxel in the multi-contrast MR image data based on the abnormality region and the model; and
creating an abnormality map of the subject's brain based on the novelty score computed for each voxel in the multi-contrast MR image data.

15. The article of manufacture of claim 14, wherein the abnormality region is defined by bounding box manually drawn by a user.

16. The article of manufacture of claim 15, wherein the abnormality region is defined by bounding box automatically defined using an unsupervised change detection method that searches for a most dissimilar region left and right halves of the subject's brain.

17. The article of manufacture of claim 15, wherein the abnormality region is defined using a fully automated procedure that analyzes the multi-contrast MR image data and generates a list of voxels that are suspected to be abnormal.

18. The article of manufacture of claim 17, wherein the fully automated procedure comprises:
fitting a Gaussian mixture model (GMM) via expectation maximization (EM) to the multi-contrast MR image data over a plurality of iterations, wherein each voxel of the multi-contrast MR image data is checked during each iteration of the fully automated procedure to determine whether it should be placed in the abnormality region or the healthy region.

19. The article of manufacture of claim 14, wherein the novelty score is computed for each voxel using an analytical multivariate extreme value theory (EVT) approximation.

20. A system for identifying abnormalities in Magnetic Resonance (MR) brain image data, the system comprising:
an imaging device configured to acquire multi-contrast MR image data of a subject's brain; and
a computer comprising one or more processors configured to:
identify, within the multi-contrast MR image data, (i) an abnormality region comprising one or more suspected abnormalities and (ii) a healthy region comprising healthy tissue,
create a model of the healthy region,
compute a novelty score for each voxel in the multi-contrast MR image data based on the abnormality region and the model, and
create an abnormality map of the subject's brain based on the novelty score computed for each voxel in the multi-contrast MR image data.
